(19) United States
(12) Patent Application Publication
(10) Pub. No.: US 2020/0297620 A1
(43) Pub. Date: Sep. 24, 2020

(54) STABLE PHARMACEUTICAL COMPOSITIONS FOR TOPICAL ADMINISTRATION AND USES THEREOF

(71) Applicant: Vidac Pharma Ltd., Jerusalem (IL)

(72) Inventors: Paul SALAMA, ASHDOD (IL); Oren Menahem BECKER, MEVASSERET ZION (IL); Chaim MANN BRICKMAN, RA'ANANA (IL)

(73) Assignee: Vidac Pharma Ltd, Jerusalem (IL)

(21) Appl. No.: 16/088,833

(22) PCT Filed: Mar. 28, 2017
(86) PCT No.: PCT/IL2017/050381

§ 371 Date: Sep. 27, 2018

Related U.S. Application Data
(60) Provisional application No. 62/313,836, filed on Mar. 28, 2016.

(51) Int. Cl. A61K 9/00; A61K 31/47

(52) U.S. Cl. CPC A61K 9/0014; A61K 9/06; A61K 31/47

(57) ABSTRACT

The present invention relates to novel pharmaceutical compositions for topical administration of a compound Formula I and methods of treating or preventing cancer or pre-cancerous conditions or benign hyperproliferative disorders, by administering the composition to a subject in need thereof. In particular, the present invention provides a stable formulation of active a compound of Formula I useful for the treatment of disorders such as actinic keratosis.

### FIELD OF THE INVENTION

The present invention provides stable topical pharmaceutical compositions comprising a compound of Formula I (Compound I), and methods of using these compositions, especially for treating or preventing cancers or pre-cancerous conditions, or benign hyperproliferative disorders, such as actinic keratosis.

### BACKGROUND OF THE INVENTION

Jasmonates are a family of plant stress hormones, which are released in instances of extreme UV radiation, osmotic shock, heat shock, pathogen attack and the like, to initiate various cascades. The use of jasmonates for the treatment of mammalian cancer has been disclosed in International Patent Application WO 02/080890 and in U.S. Pat. No. 6,469,061 wherein the jasmonates were shown to induce direct cytotoxicity for various types of human cancer cells derived from breast, prostate, skin, and blood cancers. Methyl jasmonate was shown to be effective in preventing development of lymphomas in mice (Fingrut and Flescher, Leukemia, 16: 608-616, 2002).

International Patent Application WO 2005/054172 discloses halogenated jasmonate derivatives, pharmaceutical compositions comprising the derivatives, and their use in reducing cancer cell growth and in treating cancer.

International Patent Applications WO 2007/066336 and WO 2007/066337 disclose jasmonate derivatives, pharmaceutical compositions comprising same, and use thereof in reducing cancer cell growth and the treatment of cancer.

International Patent Applications WO 2010/143180 discloses jasmonate derivatives, an assay for determining the therapeutic effect of a jasmonate ester derivative in benign hyperproliferative skin disorders, pharmaceutical compositions comprising jasmonate derivatives, and use thereof in treating benign hyperproliferative disorders of the skin, in particular, actinic keratosis.

Compounds comprising an ester group are prone to hydrolysis, which constitutes an obstacle in the preparation of a suitable formulation which maintains the structural integrity of the active compound. There is an unmet need for stable pharmaceutical compositions comprising such compounds, especially a compound of Formula I as described herein. Such compounds are useful for the treatment or prevention of cancer or pre-cancerous conditions, or benign hyperproliferative disorders.

### SUMMARY OF THE INVENTION

The present invention provides novel formulations for topical administration of a compound represented by the structure of Formula I (Compound I), and pharmaceutically acceptable excipients.

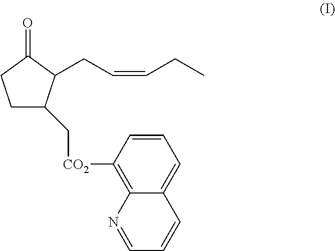

The formulations of the invention are specifically designed to stabilize Compound I, which contains an ester group that is prone to lysis in nucleophilic media (e.g., water, alcohols). As demonstrated herein, conventionally used topical excipients could not preserve the stability of Compound I and demonstrated poor chemical compatibility and degradation of the active ester component. It has now been surprisingly discovered that a specific combination of excipients can provide stable formulations containing Compound I for an improved therapeutic benefit by topical administration. These excipients are devoid of hydroxyl (OH) or other nucleophilic functionalities, and are mainly non-polar compounds that are compatible with the lipophilic nature of Compound I.

Thus, the present invention provides, in one aspect, a topical pharmaceutical composition comprising a therapeutically effective amount of a compound represented by the structure of Formula I, or a pharmaceutically acceptable salt or solvate thereof, and at least one pharmaceutically acceptable excipient selected from the group consisting of a glycol diester with a saturated or unsaturated fatty acid, a triglyceride, an aprotic organic solvent, and any combination thereof. In one embodiment, at least one of the excipients is a non-aqueous excipient. Optionally, the composition further comprises an oil, a wax, or a combination thereof.

In one embodiment, the pharmaceutically acceptable excipient of the composition described above comprises a compound represented by the structure:

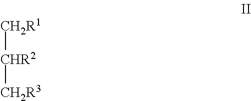

wherein:

R¹, R² and R³ are each selected from the group consisting of H and R⁴—COO—, wherein at least two of R′, R² and R³ are R⁴—COO; and

R⁴—COO— is the residue of a saturated or unsaturated fatty acid.

In one embodiment, the pharmaceutically acceptable excipient comprises a triglyceride. In some embodiments, the triglyceride comprises medium chain (e.g., C₈-C₁₂) saturated or unsaturated fatty acids. In one embodiment, the pharmaceutically acceptable excipient is caprylic/capric triglyceride (in one embodiment, Labrafac lipophlie WL 1349).

In another embodiment, the pharmaceutically acceptable excipient comprises a glycol diester. In some embodiments, the glycol diester is a propylene glycol diester with saturated or unsaturated fatty acids. In one embodiment, the fatty acid is a medium chain (e.g., C₈-C₁₂) saturated or unsaturated fatty acid. In one embodiment, the pharmaceutically acceptable excipient is propylene glycol dicaprylate (in one embodiment, Labrafac PG).

In some embodiments, the pharmaceutically acceptable excipient comprises a mixture of a triglyceride and a propylene glycol diester with saturated or unsaturated fatty acids.

In one embodiment, the pharmaceutically acceptable excipient is an organic solvent, which, in one embodiment, is dimethylsulfoxide (DMSO).

In another embodiment, the composition of the invention further comprises an oil, a wax or a combination thereof. Each possibility represents a separate embodiment of the present invention.

In some embodiments, the composition of the invention comprises the compound of Formula I, propylene glycol dicaprylate, caprylic/capric triglyceride, dimethylsulfoxide (DMSO), petrolatum and paraffin wax.

In some embodiments, the composition of the invention comprises a compound of Formula I in an amount of from about 5% w/w to about 30% w/w. In some embodiments, the composition of the invention comprises a compound of Formula I in an amount of about 5% w/w. In some embodiments, the composition of the invention comprises a compound of Formula I in an amount of about 10% w/w. In some embodiments, the composition of the invention comprises a compound of Formula I in an amount of about 15% w/w. In some embodiments, the composition of the invention comprises a compound of Formula I in an amount of about 20% w/w.

In some embodiments, the composition of the invention comprises: a compound of Formula I in an amount of from about 5% to about 30% w/w; propylene glycol dicaprylate in an amount of from about 1% to about 10% w/w; caprylic/capric triglyceride in an amount of from about 30% to about 50% w/w; dimethyl sulfoxide in an amount of from about 0% to about 10% w/w; petrolatum in an amount of from about 20% to about 50% w/w; and paraffin wax in an amount of from about 0 to about 10% w/w. The term “w/w” denotes weight/weight.

In other embodiments, the composition of the invention comprises: a compound of Formula I in an amount of about 5% w/w; propylene glycol dicaprylate in an amount of about 10% w/w; caprylic/capric triglyceride in an amount of about 50% w/w; dimethyl sulfoxide in an amount of about 1% w/w; petrolatum in an amount of about 30% w/w; and paraffin wax in an amount of about 4% w/w.

In other embodiments, the composition of the invention comprises: a compound of Formula I in an amount of about 10% w/w; propylene glycol dicaprylate in an amount of about 7.5% w/w; caprylic/capric triglyceride in an amount of about 47.5% w/w; dimethyl sulfoxide in an amount of about 1% w/w; petrolatum in an amount of about 30% w/w; and paraffin wax in an amount of about 4% w/w.

In other embodiments, the composition of the invention comprises: a compound of Formula I in an amount of about 15% w/w; propylene glycol dicaprylate in an amount of about 5% w/w; caprylic/capric triglyceride in an amount of about 45% w/w; dimethyl sulfoxide in an amount of about 1% w/w; petrolatum in an amount of about 30% w/w; and paraffin wax in an amount of about 4% w/w.

In other embodiments, the composition of the invention comprises: a compound of Formula I in an amount of about 20% w/w; propylene glycol dicaprylate in an amount of about 2.5% w/w; caprylic/capric triglyceride in an amount of about 42.5% w/w; dimethyl sulfoxide in an amount of about 1% w/w; petrolatum in an amount of about 30% w/w; and paraffin wax in an amount of about 4% w/w.

In some embodiments, the topical pharmaceutical compositions are formulated as an ointment, cream or gel. Each possibility represents a separate embodiment of the present invention. In one embodiment, the composition is in the form of an ointment.

In some embodiments, the composition described above is essentially free of water, alcoholic solvents and carboxylic acids.

In another aspect, the present invention further provides a composition as described above for use in treating or preventing cancer, a pre-cancerous condition or a benign hyperproliferative disorder.

In yet another aspect, the present invention further provides a method of treating or preventing cancer or a pre-cancerous condition, comprising the step of administering to a subject in need thereof the composition described above.

In some embodiments, the methods of the present invention comprise a step of administering the composition as described herein in the form of an ointment, cream or gel. In certain embodiments, the composition is administered in the form of an ointment. In some embodiments, the composition is administered one time per day. In other embodiments, the composition is administered 2, 3, or 4 times per day. In some embodiments, the composition is administered once every 2 days, or once every 3 days, or once every 4 days, or once every 5 days, or once every 6 days, or once every 7 days, or once every 8 days, or once every 9 days, or once every 10 days. In other embodiments, the composition is administered once every week, or once every 2 weeks, or once every 3 weeks, or once every 4 weeks, or once every 5 weeks, or once every 6 weeks, or once every 7 weeks, or once every 8 weeks.

In one embodiment, the compound of Formula I is useful for treating actinic keratosis or psoriasis.

In some embodiments, the cancer is skin cancer selected from melanoma and non-melanoma skin cancer. In some embodiments, the non-melanoma skin cancer is selected from basal cell carcinoma (BCC) and squamous cell carcinoma (SCC).

In some embodiments, the composition can be administered in combination with at least one other active agent.

### DETAILED DESCRIPTION OF THE INVENTION

The present invention relates to compositions comprising a compound of Formula I and methods of use thereof in treating or preventing cancer, pre-cancerous conditions, or benign hyperproliferative disorders.

The compound of Formula I comprises an ester moiety, which is prone to lysis upon contact with protic environment such as water and alcoholic solvents. Thus, many conventionally used excipients useful for topical formulations are not suitable for use in combination with Compound I as the latter is unstable and decomposes in their presence. The present invention provides uniquely designed formulations comprising pharmaceutical excipients that are suitable for use in topical compositions on the one hand, while preserving the stability of the compound of Formula I on the other. Such formulations contain excipients that are compatible with the lipophilic nature of Compound I. The formulations of the invention have utility for preventing cancer, pre-cancerous conditions, and benign hyperproliferative disorders.

In accordance with the principles of the present invention, Compound I is mixed with stabilizing excipients useful for topical administration in a formulation which is essentially free of compounds bearing a nucleophilic character like water, alcoholic solvents and carboxylic acids. In some embodiments, the topical pharmaceutical composition of the present invention comprises a therapeutically effective amount of a compound represented by the structure of Formula I, or a salt or solvate thereof, and at least one pharmaceutically acceptable excipient selected from the group consisting of a glycol diester with a saturated or unsaturated fatty acid, a triglyceride, an aprotic organic solvent, and any combination thereof, and optionally an oil and/or a wax

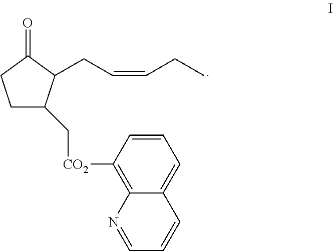

In one embodiment, at least one of the excipients is a non-aqueous excipient.

In accordance with the principles of the present invention, Compound I is mixed with stabilizing excipients useful for topical administration in a formulation which is essentially free of compounds bearing a nucleophilic character like water, alcoholic solvents and carboxylic acids. In some embodiments, the topical pharmaceutical composition of the present invention comprises a therapeutically effective amount of a compound represented by the structure of Formula I, or a pharmaceutically acceptable salt or solvate thereof, and at least one pharmaceutically acceptable excipient selected from the group consisting of a glycol diester with a saturated or unsaturated fatty acid, a triglyceride, an aprotic organic solvent, and any combination thereof, and optionally an oil and/or a wax

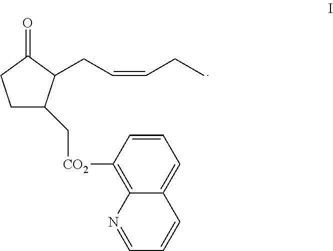

In one embodiment, the pharmaceutically acceptable excipient comprises a compound represented by the structure:

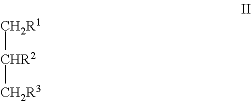

wherein:

R¹, R² and R³ are each selected from the group consisting of H and R⁴—COO—, wherein at least two of R¹, R² and R³ are R⁴—COO; and

R⁴—COO— is the residue of a saturated or unsaturated fatty acid.

In another embodiment, the pharmaceutically acceptable excipient comprises a triglyceride of Formula III or a combination thereof

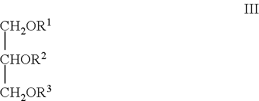

wherein

R¹, R² and R³ are each R⁴—C(═O)—; and R⁴—C(═O)O— is the residue of an unsaturated or saturated fatty acid.

In some embodiments, the pharmaceutically acceptable excipient comprises a triglyceride with a medium chain (e.g., C₈-C₁₂) saturated or unsaturated fatty acid. In one embodiment, the pharmaceutically acceptable excipient is caprylic/capric triglyceride (in one embodiment Labrafac lipophile WL 1349).

In another embodiment, the pharmaceutically acceptable excipient comprises a propylene glycol diester with a saturated or unsaturated fatty acid. In one embodiment, the fatty acid is a medium chain (e.g., C₈-C₁₂) saturated or unsaturated fatty acid. In one embodiment, the pharmaceutically acceptable excipient is propylene glycol dicaprylate (in one embodiment, Labrafac PG).

In one embodiment, the organic solvent is dimethylsulfoxide (DMSO).

The present invention may further comprise optional components such as oils or waxes so as to provide a semi-solid consistency as desired. Examples of such optional components are oils and/or waxes. Additional inactive excipients such as preservatives, anti-oxidants, perfumes and the like can also be added.

Oils that can be used as optional components in the compositions of the present invention include, but are not limited to, petrolatum (petroleum jelly), vegetable oil, fruit oil, plant oil, animal oil, mineral oil, coconut oil, olive oil, lanolin, peanut oil, hydrogenated and sulphated oils such as cotton seed oil, soybean oils, almond oil, sesame oil, and the like.

Waxes that can be used as optional components in the compositions of the present invention include, but are not limited to, paraffin wax, beeswax, carnauba wax, cetyl ester wax, microcrystalline wax, spermaceti wax, and the like.

In some embodiments, the composition of the invention comprises the compound represented by the structure of Formula I, propylene glycol dicaprylate (Labrafac PG), caprylic/capric triglyceride (Labrafac lipophile WL 1349), dimethylsulfoxide (DMSO), petrolatum and paraffin wax.

In some embodiments, the composition of the invention comprises a compound of Formula I in an amount of from about 5% w/w to about 30% w/w. In some embodiments, the composition of the invention comprises a compound of Formula I in an amount of about 5% w/w. In some embodiments, the composition of the invention comprises a compound of Formula I in an amount of about 10% w/w. In some embodiments, the composition of the invention comprises a compound of Formula I in an amount of about 15% w/w. In some embodiments, the composition of the invention comprises a compound of Formula I in an amount of about 20% w/w.

In some embodiments, the composition of the invention comprises a compound of Formula I in an amount of from about 5% to about 30% w/w; propylene glycol dicaprylate in an amount of from about 1% to about 10% w/w; caprylic/capric triglyceride in an amount of from about 30% to about 50% w/w; dimethyl sulfoxide in an amount of from about 0% to about 10% w/w; petrolatum in an amount of from about 20% to about 50% w/w; and paraffin wax in an amount of from about 0 to about 10% w/w.

In some embodiments, the composition of the invention comprises a compound of Formula I in an amount of about 5% w/w; propylene glycol dicaprylate in an amount of about 10% w/w; caprylic/capric triglyceride in an amount of about 50% w/w; dimethyl sulfoxide in an amount of about 1% w/w; petrolatum in an amount of about 30% w/w; and paraffin wax in an amount of about 4% w/w.

In other embodiments, the composition of the invention comprises a compound of Formula I in an amount of about 10% w/w; propylene glycol dicaprylate in an amount of about 7.5% w/w; caprylic/capric triglyceride in an amount of about 47.5% w/w; dimethyl sulfoxide in an amount of about 1% w/w; petrolatum in an amount of about 30% w/w; and paraffin wax in an amount of about 4% w/w.

In other embodiments, the composition of the invention comprises: a compound of Formula I in an amount of about 15% w/w; propylene glycol dicaprylate in an amount of about 5% w/w; caprylic/capric triglyceride in an amount of about 45% w/w; dimethyl sulfoxide in an amount of about 1% w/w; petrolatum in an amount of about 30% w/w; and paraffin wax in an amount of about 4% w/w.

In certain embodiments, the composition of the invention comprises a compound of Formula I in an amount of about 20% w/w; propylene glycol dicaprylate in an amount of about 2.5% w/w; caprylic/capric triglyceride in an amount of about 42.5% w/w; dimethyl sulfoxide in an amount of about 1% w/w; petrolatum in an amount of about 30% w/w; and paraffin wax in an amount of about 4% w/w.

In some embodiments, the topical pharmaceutical compositions are formulated as an ointment, cream or gel. Each possibility represents a separate embodiment of the present invention. In one embodiment, the composition is in the form of an ointment.

In some embodiments, the composition described above is essentially free of water, alcoholic solvents and carboxylic acids.

The pharmaceutically acceptable excipients according to the principles of the present invention may be a triglyceride, a glycol diester or a combination thereof In some embodiments, the triglyceride and/or the glycol diester comprise saturated or unsaturated fatty acids. The term “saturated fatty acid” encompasses a carboxylic acid with an elongated aliphatic saturated chain. The term “unsaturated fatty acid” encompasses a carboxylic acid with an elongated aliphatic chain which have one or more double bonds between the carbon atoms. The length of the carbon chain may vary, but may generally be between 4 and 30 carbon atoms (C₄-C₃₀). In some embodiments, the triglyceride and/or glycol diester comprises medium chain (e.g., C₈-C₁₂) saturated or unsaturated fatty acids.

Compound I may be present in the form of a salt. The term “salt” encompasses both basic and acid addition salts, and include salts formed with the organic and inorganic anions and cations discussed below. Examples of acid addition salts include hydrochloric, hydrofluoric, trifluoroacetic, sulfuric, phosphoric, acetic, succinic, citric, lactic, maleic, fumaric, palmitic, cholic, pamoic, mucic, D-glutamic, D-camphoric, glutaric, phthalic, tartaric, lauric, stearic, salicylic, methanesulfonic, benzenesulfonic, sorbic, picric, benzoic, cinnamic, and like acids.

The present invention also includes the use of solvates of Compound I and salts thereof “Solvate” means a physical association of a compound of the invention with one or more solvent molecules. This physical association involves varying degrees of ionic and covalent bonding, including hydrogen bonding. In certain instances, the solvate will be capable of isolation. “Solvate” encompasses both solution-phase and isolatable solvates.

### Therapeutic Use

In one aspect, the present invention provides a method of treating or preventing cancer or a pre-cancerous condition or a benign hyperproliferative disorder, by administering to a subject a pharmaceutical composition as described above.

In another aspect, the present invention further provides the use of the pharmaceutical composition as described above in treating or preventing cancer, a pre-cancerous condition or a benign hyperproliferative disorder.

In some embodiments, the compound of Formula I is useful for treating psoriasis.

In some embodiments, the compound of Formula I is useful for treating actinic keratosis.

In other embodiments, the cancer is skin cancer selected from melanoma and non-melanoma skin cancer. Each possibility represents a separate embodiment of the present invention.

In other embodiments, the non-melanoma skin cancer is selected from basal cell carcinoma (BCC) and squamous cell carcinoma (SCC). Each possibility represents a separate embodiment of the present invention.

In some embodiments, the composition can be administered in combination with at least one other active agent.

In another embodiment, the topical pharmaceutical composition comprising Compound I as described herein may be administered with a non-topical pharmaceutical composition comprising Compound I. In one embodiment, the non-topical pharmaceutical composition comprises a composition formulated for intravenous or subcutaneous administration. The non-topical pharmaceutical compositions can be, in another embodiment, administered to a subject by any method known to a person skilled in the art, such as parenteral, intramuscular, intra-dermal, intra-nasal, oral, ophthalmic, or intra-peritoneal administration. In one embodiment, the topical and non-topical pharmaceutical compositions comprising Compound I are administered simultaneously. In another embodiment, the topical and non-topical pharmaceutical compositions comprising Compound I are administered sequentially. In one embodiment, the topical formulation is administered prior to the administration of the non-topical formulation. In another embodiment, the non-topical formulation is administered prior to the administration of the topical formulation.

In some embodiments, the composition is administered one time per day. In other embodiments, the composition is administered 2, 3, or 4 times per day. For example, in some embodiments, the composition is administered 2 times per day. In some embodiments, the composition is administered one time per day. In other embodiments, the composition is administered 3 times per day. In other embodiments, the composition is administered 4 times per day.

In some embodiments, the methods of the present invention comprise administering a composition as described herein once every 2 days, once every 3 days, once every 4 days, once every 5 days, once every 6 days, once every 7 days, once every 8 days, once every 9 days, or once every 10 days.

In some embodiments, the methods of the present invention comprise administering a composition as described herein once every 2 days. In some embodiments, the composition is administered once every 3 days. In some embodiments, the composition is administered once every 4 days. In some embodiments, the composition is administered once every 5 days. In some embodiments, the composition is administered once every 6 days. In some embodiments, the composition is administered once every 7 days. In some embodiments, the composition is administered once every 8 days. In some embodiments, the composition is administered once every 9 days. In some embodiments, the composition is administered once every 10 days.

In some embodiments, the methods of the present invention comprise administering a composition as described herein once every week, once every 2 weeks, once every 3 weeks, once every 4 weeks, once every 5 weeks, once every 6 weeks, once every 7 weeks, or once every 8 weeks. In some embodiments, the composition is administered once every week. In some embodiments, the composition is administered once every 2 weeks. In some embodiments, the composition is administered once every other week. In some embodiments, the composition is administered once every 3 weeks. In some embodiments, the composition is administered once every 4 weeks. In some embodiments, the composition is administered once every 5 weeks. In some embodiments, the composition is administered once every 6 weeks. In some embodiments, the composition is administered once every 7 weeks. In some embodiments, the composition is administered once every 8 weeks.

In some embodiments, the composition is administered once every month. In some embodiments, the composition is administered once every 2 months. In some embodiments, the composition is administered once every other month. In some embodiments, the composition is administered once every 3 months. In some embodiments, the composition is administered once every 4 months. In some embodiments, the composition is administered once every 5 months. In some embodiments, the composition is administered once every 6 months. In some embodiments, the composition is administered once every 7 months. In some embodiments, the composition is administered once every 8 months. In some embodiments, the composition is administered once every 9 months. In some embodiments, the composition is administered once every 10 months. In some embodiments, the composition is administered once every 11 months. In some embodiments, the composition is administered once every 12 months.

In some embodiments, the methods of the present invention comprise administering a composition as described herein on a schedule as described hereinabove (e.g., daily, weekly, monthly) with planned or unplanned hiatuses in the regimen. According to this aspect and in one embodiment, the methods of the present invention comprise administering a composition for one week and then not administering the composition for one week. In another embodiment, the composition is administered for one week and then not administered for two weeks. In another embodiment, the composition is administered for one week and then not administered for three weeks.

In another embodiment, the composition is administered for two weeks and then not administered for one week. In another embodiment, the composition is administered for two weeks and then not administered for two weeks. In another embodiment, the composition is administered for two weeks and then not administered for four weeks.

In another embodiment, the composition is administered for one month and then not administered for one month. In another embodiment, the composition is administered for one month and then not administered for two months. In another embodiment, the composition is administered for one month and then not administered for three months.

In another embodiment, the composition is administered for two months and then not administered for one month. In another embodiment, the composition is administered for two months and then not administered for two months. In another embodiment, the composition is administered for two months and then not administered for four months.

In some embodiments, the methods of the present invention comprise administering a composition as described herein for a total duration of 1-3 months. In one embodiment, the treatment regimen lasts up to 28 days. In another embodiment, the treatment regimen lasts up to 56 days. In another embodiment, the treatment regimen lasts up to 84 days.

In some embodiments, the methods of the present invention comprise administering a composition as described herein for a total duration of up to 1 week. In another embodiment, the compositions are administered for up to 2 weeks. In another embodiment, the compositions are administered for up to 3 weeks. In another embodiment, the compositions are administered for up to 1 month. In another embodiment, the compositions are administered for up to 2 months. In another embodiment, the compositions are administered for up to 3 months. In another embodiment, the compositions are administered for up to 4 months. In another embodiment, the compositions are administered for up to 5 months. In another embodiment, the compositions are administered for up to 6 months. In another embodiment, the compositions are administered for up to 9 months. In another embodiment, the compositions are administered for up to 1 year. In another embodiment, the compositions are administered for up to 18 months. In another embodiment, the compositions are administered for up to 2 years.

In one embodiment, the pharmaceutical compositions comprise an effective amount of Compound I. The term “therapeutically effective amount” or “an effective amount” as used herein refers, in one embodiment, to a quantity of a compound which is sufficient to provide a beneficial effect to the subject to which the compound is administered. The effective amount, according to the principles of the present invention can be determined by any one of ordinary skill in the art and can be tested on various models both in vitro and in vivo.

The term “pharmaceutically acceptable” means in one embodiment, that the compound is approved by a regulatory agency of the Federal or a state government or listed in the U. S. Pharmacopeia or other generally recognized pharmacopeia for use in animals and, more particularly, in humans Pharmaceutically acceptable non-aqueous excipient in the context of the present invention refers to an approved ingredient which is not water or water-based material.

The term “inhibition of proliferation” in relation to cancer cells, in the context of the present invention and in one embodiment, refers to a decrease in at least one of the following: number of cells (due to cell death which may be necrotic, apoptotic or any other type of cell death or combinations thereof) as compared to control; decrease in growth rates of cells, i.e. the total number of cells may increase but at a lower level or at a lower rate than the increase in control; decrease in the invasiveness of cells (as determined for example by soft agar assay) as compared to control even if their total number has not changed; progression from a less differentiated cell type to a more differentiated cell type; a deceleration in the neoplastic transformation; or alternatively the slowing of the progression of the cancer cells from one stage to the next. In one embodiment, the methods of the present invention prevent, inhibit or suppress formation of metastases. In another embodiment, the methods of the present invention prevent, inhibit or suppress the spread of metastases. In one embodiment, the methods of the present invention prevent, inhibit or suppress the spread of metastases to other organs. In one embodiment, the methods of the present invention prevent, inhibit or suppress the spread of metastases to lymph nodes. In another embodiment, the methods of the present invention prevent, inhibit or suppress the spread of metastases below the epidermis, which in one embodiment is a dermis cell and, in another embodiment, is a subcutis cell.

As used herein, the term “administering” refers, in one embodiment, to bringing something into contact with a compound of the present invention. In one embodiment, administration may be to cells or tissue cultures, or to living organisms, for example humans. Thus, in one embodiment, the present invention encompasses administering the compounds of the present invention to a human subject. In another embodiment, the present invention encompasses administering the compounds of the present invention to a mammalian subject, which in one embodiment, is murine, canine, feline, bovine, ovine, or porcine.

As used herein, the term “subject” refers, in one embodiment, to a human subject. In another embodiment, the subject is murine, which in one embodiment, is a mouse, and, in another embodiment, is a rat. In another embodiment, the subject is canine, feline, bovine, ovine, or porcine. In another embodiment, the subject is a mammal, including primates, such as monkeys and humans, horses, cows, sheep, pigs, goats, cats, dogs, rabbits, birds such as turkey, chickens, and ducks, and rodents such as rats, mice, guinea pigs, and hamsters. In a particular embodiment, the mammal to be treated is human. Other subjects include species that are commonly used in scientific research, animal husbandry or as human companions.

The term “treating” as used herein refers, in one embodiment, to alleviation of the adverse effects of the disease or disorder, which alleviation may be manifested by a decrease in at least one of the following: reduction in the number of abnormal epidermal cells (due to cell death which may be necrotic, apoptotic or any other type of cell death or combinations thereof) as compared to control; decrease in proliferation of cells, i.e. the total number of cells may increase but at a lower level or at a lower rate than the increase in control; or decrease in the invasiveness of cells (as determined for example by soft agar assay) as compared to control even if their total number has not changed.

In one embodiment, “treating” as used herein refers to therapeutic treatment. In one embodiment, “preventing”, “suppressing” or “inhibiting” as used herein refers to prophylactic or preventative measures, wherein the object is to prevent or lessen the targeted condition or disorder as described herein. Thus, in one embodiment, “treating” refers inter alia to delaying progression, expediting remission, inducing remission, augmenting remission, speeding recovery, increasing efficacy of or decreasing resistance to alternative therapeutics or other formulations of the same drug, in one embodiment, given via another route of administration, or a combination thereof In one embodiment, “preventing” refers, inter alia, to delaying the onset of symptoms, preventing relapse to a disease, decreasing the number or frequency of relapse episodes, increasing latency between symptomatic episodes, or a combination thereof. In one embodiment, “suppressing” or “inhibiting”, refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

The term “topical” or “topical formulation” in the context of the present invention comprises a formulation that is applied directly to a particular site on or in the body. In one embodiment, topical administration comprises administration to the skin. In another embodiment, topical administration comprises administration to a mucous membrane (transmucosal). In another embodiment, topical administration comprises administration by inhalation. In another embodiment, topical administration comprises administration to conjunctiva, ear, or tooth.

In another embodiment, the pharmaceutical compositions are administered topically to body surfaces and are thus formulated in a form suitable for topical administration. In some embodiments, the methods of the present invention comprise administering a composition as described herein in the form of an ointment, cream or gel. In certain embodiments, the composition is administered in the form of an ointment. In another embodiment, lotions, drops and the like are formulated for topical administration. In another embodiment, the pharmaceutical composition is administered as a suppository, for example a rectal suppository or a urethral suppository. In another embodiment, the pharmaceutical composition is delivered in a vesicle, e.g. a liposome.

In another embodiment, the pharmaceutical compositions provided herein are controlled-release compositions, i.e., compositions in which the antigen is released over a period of time after administration. Controlled- or sustained-release compositions include formulation in lipophilic depots (e.g. fatty acids, waxes, oils). In another embodiment, the composition is an immediate-release composition, i.e., a composition in which the pharmaceutical composition is released immediately after administration.

In another embodiment, the pharmaceutical composition is delivered in a controlled release system. In another embodiment, the pharmaceutical composition is administered using a transdermal patch, liposomes, or other modes of administration. In another embodiment, polymeric materials are used; e.g. in microspheres in or an implant.

The term “cancer” in the context of the present invention, in one embodiment, includes all types of neoplasm whether in the form of solid or non-solid tumors, and includes both malignant and premalignant conditions as well as their metastasis. In one embodiment, the cancer is any cancer that may be treated with a composition as described herein. In another embodiment, the cancer is any cancer that may be treated with a topical formulation. In one embodiment, the cancer is skin cancer. In another embodiment, the cancer is breast cancer, bladder cancer, which, in one embodiment comprises non-muscle invasive bladder cancer (NMIBC), cervical cancer, anal cancer, or oral cancer, which, in one embodiment comprises oral cavity or oropharyngeal cancer.

The term “treatment of cancer” in the context of the present invention includes, in one embodiment, at least one of the following: a decrease in the rate of growth of the cancer (i.e. the cancer still grows but at a slower rate); cessation of growth of the cancerous growth, i.e., stasis of the tumor growth, and, in one embodiment, the tumor diminishes or is reduced in size. The term also includes reduction in the number of metastasis, reduction in the number of new metastasis formed, slowing of the progression of cancer from one stage to the other and a decrease in the angiogenesis induced by the cancer. In one embodiment, the tumor is totally eliminated.

The term “skin cancer” of the present invention includes, in one embodiment, melanoma and non-melanoma skin cancer. Melanoma refers, in one embodiment, to cancerous growths originate in the pigment-producing melanocytes in the basal layer of the epidermis. Non-melanoma skin cancer includes, in one embodiment, Basal Cell Carcinoma (BCC) which are abnormal, uncontrolled growths or lesions that arise in the skin's basal cells, lining the deepest layer of the epidermis (the outermost layer of the skin) and, in another embodiment, Squamous Cell Carcinoma (SCC), characterized by an uncontrolled growth of abnormal cells arising in the squamous cells, which compose most of the skin's upper layers (the epidermis). In another embodiment, the non-melanoma skin cancer comprises: angiosarcoma, cutaneous T-cell lymphoma (CTCL), dermatofibrosarcoma protuberans, Merkel cell carcinoma, and sebaceous gland carcinoma.

In another embodiment, “cancer” comprises BCC or SCC in organs other than the skin. In one embodiment, the non-skin organ comprises anus, cervix, head and neck, vagina, breast, bladder, esophagus, urinary, prostate, and lung. Each of these comprises an embodiment of the invention.

The term “pre-cancer” or “pre-malignant” as used herein interchangeably refers, in one embodiment, to diseases, syndromes or other conditions associated with an increased risk of cancer. Pre-cancer conditions in the context of the present invention include, but are not limited to: breast calcifications, vaginal intra-epithelial neoplasia, Barrett's esophagus, atrophic gastritis, dyskeratosis congenital, sideropenic dysphagia, lichen planus, oral submucous fibrosis, solar elastosis, cervical dysplasia, leukoplakia and erythroplakia. In another embodiment, the pre-cancer condition is Bowen's Disease. In another embodiment, the pre-cancer condition is squamous cell carcinoma in situ. In another embodiment, the pre-cancer condition is actinic keratosis.

The term “benign hyperproliferative disorder” as used herein refers, in one embodiment, to a condition in which there is an abnormal growth and differentiation of cells and an increase in the amount of organic tissue that results from cell proliferation. The benign hyperproliferative disorder may be attributed to lack of response or inappropriate response to regulating factors, or alternatively to dysfunctional regulating factors. Non-limiting example of benign hyperproliferative disorder are psoriasis, Lentigines (liver spots), and Nevus (mole).

The term “treating benign hyperproliferative disorders” in the context of the present invention includes, in one embodiment, at least one of the following: a decrease in the rate of growth of the lesions; or cessation of growth of the lesions characteristic of the disorder. In one embodiment, the lesions which are characteristic of the hyperproliferative disorder are diminished, reduced in size or totally eliminated. In one embodiment, the pharmaceutical composition comprising Compound I, according to the principles of the present invention, is used for the treatment of actinic keratosis. Actinic keratosis typically is a sharply outlined verrucous or keratotic growth which may become malignant.

The term “actinic keratoses” as used in the context of the present invention includes, in one embodiment, skin lesions of keratinocytes which are areas of skin in which tissue shows the tendency to develop into cancer, although the tissue in its present state is not cancerous. Epithelial lesions include actinic keratosis (also called solar keratosis or senile keratosis), hypertrophic actinic keratosis, Bowenoid actinic keratosis, arsenical keratosis, hydrocarbon keratosis, thermal keratosis, radiation keratosis, chronic scar keratosis, viral keratosis, actinic cheilitis, Bowen's disease, erythroplaquia of queyrat, oral erythroplaquia, leukoplakia, and intraepidermal epithelialoma.

The term “psoriasis” as used herein refers, in one embodiment, to a condition in which cells build up rapidly on the surface of the skin, forming thick, silvery scales and itchy, dry, red patches or plaques that are sometimes painful. The patches may vary in severity from small and localized to complete body coverage. Injury to the skin can trigger psoriatic skin changes at that spot, which is known as the Koebner phenomenon. In one embodiment, psoriasis is an autoimmune condition. In one embodiment, psoriasis comprises plaque psoriasis, guttate psoriasis, inverse psoriasis, pustular psoriasis, and erythrodermic psoriasis.

The term “treating psoriasis” in the context of the present invention includes, in one embodiment, decreased plaque area, decreased skin cell proliferation, decreased scaliness, decreased pruritus (itchiness), or decreased pain. In one embodiment, the plaques or patches which are characteristic of psoriasis are diminished, reduced in size or totally eliminated. In one embodiment, the pharmaceutical composition comprising Compound I, according to the principles of the present invention, is used for the treatment of psoriasis. In another embodiment, the pharmaceutical composition comprising Compound I is used for the prevention, inhibition or suppression of psoriasis, which in one embodiment comprises decreased frequency or severity of flare-ups or increased remission periods.

In some embodiments, the term “Formula I ointment” refers to the composition of the invention. In other embodiments, the term “Formula I ointment” refers to the composition or the formulation as described in Example 2. In some embodiments, the term “Formula I ointment” includes 5% Formula I, 10% Formula I, 15% Formula I, and 20% Formula I. In some embodiments, the terms “5% Formula I” and “Formula I (5%)” are used interchangeably herein;

the terms “10% Formula I” and “Formula I (10%)” are used interchangeably herein; the terms “15% Formula I” and “Formula I (15%)” are used interchangeably herein; and the terms “20% Formula I” and “Formula I (20%)” are used interchangeably herein.

### LIST OF ABBREVIATIONS

AE Adverse event
AK Actinic keratosis
BMI Body mass index
CSR Clinical study report
IEA Interim Efficacy Analysis
IND Investigational new drug
LLOQ Lower limit of quantification
LSR Local Skin Reaction
PK Pharmacokinetics
SAE Serious adverse event
TEAE Treatment emergent adverse event

The invention will be further illustrated with reference to the following illustrative examples, which are not intended to limit the scope of the invention in any manner.

### EXAMPLES

### Example 1: Accelerated Single Combination Stability Study

Topical formulation of Compound I was carefully designed while considering the chemical sensitivity of Compound I. The stability of Compound I was studied in the presence of inactive ingredients suitable for topical formulation, while water and alcohols which may serve as potential nucleophiles were excluded. Ingredients suitable for being carriers, penetration enhancers, or thickening agents were pre-selected and tested in combination with Compound I: Labrafac PG (propylene glycol dicaprylate); Labrafac lipophile WL1349 (Caprylic/capric triglyceride); Gelucire 43/01 (glyceride and PEG esters); (+)-Limonene; PEG 400 Dioleate; PEG 400 Dilaurate; Oleic acid and DMSO (dimethyl sulfoxide).

A short accelerated single combination (Compound I+1 excipient) stability study was performed in 40° C. and 65% relative humidity (RH).

The amount of Compound I was measured at the following time points: time zero, three days, one week and two weeks, in order to evaluate the compatibility of Compound I with the pre-selected ingredients (Table 1).

TABLE 1
Compatibility of Compound I with various excipients

| | Compound I % of declared amount | | | |
|---|---|---|---|---|
| Excipients Name | Time zero | 3 days | 1 week | 2 weeks |
| Labrafac PG (Propylene | 97.2 | 97.5 | 96.8 | 92.3 |
| glycol dicaprylate) | | | | |
| Labrafac Lipophile WL | 97.3 | 97.2 | 97.6 | 94.1 |
| 1349 (Caprylic/capric | | | | |
| triglyceride) | | | | |
| Gelucire 43/01 | 89.1 | 80.3 | 85.7 | 76.0 |
| R-(+)-Limonene | 98.3 | 92.7 | 82.8 | 72.9 |
| PEG 400 Dioleate (DO) | 96.4 | 93.5 | 90.1 | 81.1 |
| PEG 400 Dilaurate (DL) | 96.9 | 94.4 | 89.5 | 80.3 |
| Oleic acid | 95.0 | 48.9 | 31.4 | 19.6 |
| DMSO | 97.6 | 96.9 | 97.1 | 96.5 |

Results: As shown in Table 1, has been surprisingly found that propylene glycol dicaprylate and caprylic/capric triglyceride are compatible with Compound I in topical formulations, while Compound I was not sufficiently stable in the presence of glyceride/PEG ester mixtures (Gelucire 43/01), PEG esters and the hydrophobic hydrocarbon Limonene. Furthermore, the presence of oleic acid resulted in a sharp degradation of Compound I.

Labrafac PG, Labrafac lipophile WL1349 and DMSO were selected out of this group with the anticipated choice of +5° C. as the normal condition storage temperature for the drug product.

### Example 2: Topical Composition

The three inactive ingredients that were found to stabilize active Compound I are liquids at room temperature, thus white Petrolatum and paraffin wax were introduced in order to get the consistency of a semi-solid ointment, giving rise to the following stable ointment formulation of Compound I:

TABLE 2
Composition of 5%, 10%, 15%, and 20% Compound I ointment

| | | | *IID limit |
|---|---|---|---|
| | Equivalent | % Used | for topical |
| Commercial name | IID name | (w/w) | % (w/w) |
| API: Compound I | N/A | 5, 10, 15, 20 | N/A |
| Labrafac PG | Propylene glycol | 10, 7.5, | 10 |
| | dicaprylate | 5, 2.5 | |
| Labrafac lipophile WL | Caprylic/capric | 50, 47.5, | 50 |
| 1349 | triglyceride | 45, 42.5 | |
| DMSO | Dimethyl sulfoxide | 1 | 45 |
| White petrolatum | Petrolatum | 30 | 99.98 |
| Paraffin wax | Paraffin white soft | 4 | 15 |
| SUM | | 100 | |

*FDA's database on Inactive Ingredients (IID)

Inactive ingredients for the ointment formulation of Compound I of the present invention were selected from the FDA's database on Inactive Ingredients (IID) used in approved topical (dermal) use.

Example 3: Phase 1 Study (STUDY A)

### STUDY A: Phase 1 Dose-Escalation Study

### Title of Study

A Phase 1 randomized, double-blinded, placebo-controlled, dose-escalation study in healthy older-adult volunteers to evaluate the safety, tolerability, and pharmacokinetics of a single topical dermal application of Formula I ointment.

### Purpose of Study

Safety, tolerability and pharmacokinetics (PK) of Formula I ointment.

### Study Objectives

To evaluate the systemic and local tolerability and safety of a single topical skin application of Formula I ointment in healthy older-adult volunteers.

To determine the plasma PK profile of the main metabolite of Formula I following a single topical skin application of Formula I ointment in healthy older=adult volunteers.

### Subject Population(s) Under Study

Healthy volunteers, 35 to 70 years of age at the time of screening, with Fitzpatrick skin types I, II, or III, who had at least one 25 cm² area of healthy skin on the forehead (pre-defined criteria).

### Treatment Regimen

A 25 cm² on the forehead was selected as the treatment field.

Subjects were sequentially assigned to 1 of 3 consecutive treatment cohorts (5%, 10%, 15%, or 20% Formula I, respectively). Five subjects were randomized in a double-blind fashion in each dose cohort with 4 subjects to receive active Formula I ointment and 1 subject to receive matched placebo (0% Formula I; vehicle control). Thus, a total of 12 subjects were allocated to receive active Formula I ointment and 3 subjects were allocated to receive placebo. The clinical trial included 3 study periods: Screening, Treatment, and Observation Periods.

### Subject Enrollment Status

1. Cumulative Enrollment Summary

TABLE 3
Cumulative Enrollment Summary in STUDY A

| Enrollment Parameter | Total |
|---|---|
| Total planned accrual: | 15 |
| Number of subjects enrolled: | 15 |
| Number of subjects completed: | 15 |
| Number of subjects that withdrew/withdrawn | 0 |
| from study: | |

2. Total Number of Enrolled Subjects by Age, Gender, and Race

TABLE 4
Subject Listing by Age, Gender, and Race in STUDY A

| Subject | | Date of informed | | | |
|---|---|---|---|---|---|
| ID | Dose group | consent | Sex | Race | Age, years |
| 10101 | Formula I | 9 Mar. 2016 | M | Caucasian | 48.7 |
| | (5%) | | | | |
| 10102 | Formula I | 10 Mar. 2016 | M | Caucasian | 46.8 |
| | (5%) | | | | |
| 10103 | Formula I | 10 Mar. 2016 | M | Caucasian | 57.3 |
| | (5%) | | | | |
| 10104 | Formula I | 11 Mar. 2016 | M | Caucasian | 56.8 |
| | (5%) | | | | |
| 10105 | Placebo | 9 Mar. 2016 | F | Caucasian | 65.7 |
| 10106 | Formula I | 18 Mar. 2016 | M | Caucasian | 42.9 |
| | (10%) | | | | |
| 10107 | Placebo | 21 Mar. 2016 | M | Caucasian | 41.2 |
| 10108 | Formula I | 1 Apr. 2016 | F | Caucasian | 69.4 |
| | (10%) | | | | |
| 10109 | Formula I | 31 Mar. 2016 | M | Caucasian | 65.6 |
| | (10%) | | | | |
| 10110 | Formula I | 5 Apr. 2016 | M | Caucasian | 35.6 |
| | (10%) | | | | |
| 10111 | Formula I | 11 Apr. 2016 | M | Caucasian | 43.1 |
| | (20%) | | | | |
| 10112 | Formula I | 18 Apr. 2016 | M | Caucasian | 56.7 |
| | (20%) | | | | |
| 10113 | Formula I | 19 Apr. 2016 | M | Caucasian | 54.3 |
| | (20%) | | | | |
| 10114 | Placebo | 19 Apr. 2016 | F | Caucasian | 49.9 |
| 10115 | Formula I | 3 May. 2016 | F | Caucasian | 61.9 |
| | (20%) | | | | |

TABLE 5
Number of Enrolled Subjects by Age,
Gender, and Race in STUDY A

| Demographic | | Total (N = 15) | |
|---|---|---|---|
| parameter | Category | n (%) | |
| Sex | Female | 4 | (26.7) |
| | Male | 11 | (73.3) |
| Age | <35 years | 0 | |
| | 35-40 years | 1 | (6.7) |
| | 41-50 years | 6 | (40.0) |
| | 51-60 years | 4 | (26.7) |
| | 61-70 years | 4 | (26.7) |
| Female by age group | <35 years | 0 | |
| (n = 4) | 35-40 years | 0 | |
| | 41-50 years | 1 | (25.0) |
| | 51-60 years | 0 | |
| | 61-70 years | 3 | (75.0) |
| Male by age group | <35 years | 0 | |
| (n = 11) | 35-40 years | 1 | (9.1) |
| | 41-50 years | 5 | (45.5) |
| | 51-60 years | 4 | (36.4) |
| | 61-70 years | 1 | (9.1) |
| Race | Caucasian | 15 | (100) |

### STUDY A Results

### Demographics and Disposition

A total of 15 subjects were enrolled in this study (12 active and 3 placebo subjects) and no subject discontinued prematurely. Generally, treatment groups were similar in terms of demographic characteristics except for gender. Two of 3 placebo treated subjects were female; no female subject received Formula I (5%); 1 female subject received Formula I (10%) and 1 female subject received Formula I (20%). All subjects were Caucasian. Subject ages ranged between 35.6 and 69.4 years. The mean age of the Formula I ointment-treated subjects was 53.3 years and the mean age of the pooled placebo-treated subjects was 52.3 years. The mean BMI value for all Formula I ointment treated subjects was 30.1 kg/m² (range between 24.5 and 34.3 kg/m²) and for the pooled placebo-treated subjects was 28.5 kg/m² (range between 22.9 and 33.1 kg/m²).

### Pharmacokinetics

Plasma concentrations of Formula I (LLOQ of 1 ng/mL) and jasmonic acid (LLOQ of 50 ng/mL) at all time points were below the LLOQ. Thus, no PK analyses were performed.

### Safety

Exposure and Dosing: Each subject received their assigned treatment. Subjects in the Formula I (5%) cohort were inadvertently under-dosed (mean amount of study drug was 195 mg), while the mean amount for the Formula I 10% and 20% cohorts was 254 mg and 247 mg, respectively (target amount was 250 mg).

Adverse Events (AEs): There were no deaths, serious adverse events (SAEs), or early study discontinuations due to AEs in this study. There were a total of 6 treatment-emergent AEs (TEAEs) reported. All the AEs were mild in severity. There were no dose-related or temporally-related patterns of AEs.

The only AE deemed related to the study drug by the investigator was in a single subject in Cohort 2 (Formula I (10%)). The subject reported mild “left eye pain” approximately 4 hours after the study drug was applied to the forehead (subject received Formula I (10%)). This event was short lived and resolved without sequelae.

Clinical Laboratory Evaluation: There were no clinically significant laboratory abnormalities or changes in laboratory parameters for any subject at any time point.

Vital Signs, Physical Findings, and Other Observations Related to Safety: There were no clinically significant changes in vital signs, physical examinations, or ECGs for any individual subject at any time point regardless of treatment.

Examinations and rating of the Treatment Field for each subject during the 24-hour treatment period and for up to 7 days after the study drug application were performed for signs of erythema, edema, weeping/exudate, vesicles, erosion/ulceration, flaking/scaling/dryness, scabbing/crusting, itching, or pain. There were no treatment emergent findings for any subject for any assessment with the exception of mild flaking/scaling/dryness on Day 3 for a single subject receiving Formula I (20%) and mild pain on Day 7 for a single subject receiving placebo.

### Progress Information

STUDY A (conducted in healthy subjects) has been completed and the treatment code has been unblinded.

### Most Frequent Adverse Events

TEAEs were reported for 4 (33.3%) subjects in any Formula I ointment group and 2 (66.7%) subjects in the placebo group (Table 6). No SAE was reported during the study. The frequencies of TEAEs by system organ class and preferred term are summarized in Table 7.

TABLE 6
Summary of Adverse Event Frequency by Treatment in STUDY A

| | Treatment | | | |
|---|---|---|---|---|
| | Pooled | Formula I | Formula I | Formula I |
| Category | Placebo | (5%) | (10%) | (20%) |
| n (%) | (N = 3) | (N = 4) | (N = 4) | (N = 4) |
| Any AE | 2 (67%) | 2 (50%) | 2 (50%) | 0 |
| Any TEAE | 2 (67%) | 2 (50%) | 1 (25%) | 0 |
| Any TEAE related to | 0 | 0 | 1 (25%) | 0 |
| treatment | | | | |
| Any SAE | 0 | 0 | 0 | 0 |

TABLE 7
Summary of the Frequency of TEAEs by System Organ
Class and Preferred Term by Treatment in STUDY A

| | Treatment | | | |
|---|---|---|---|---|
| System Organ Class | Pooled | Formula | Formula | Formula |
| Preferred Term | Placebo | I (5%) | I (10%) | I (20%) |
| n (%) | (N = 3) | (N = 4) | (N = 4) | (N = 4) |
| Any TEAE | 2 (67%) | 2 (50%) | 1 (25%) | 0 |
| Eye disorders | 0 | 1 (25%) | 1 (25%) | 0 |
| Eye irritation | 0 | 1 (25%) | | 0 |
| Eye pain | 0 | 0 | 1 (25%) | 0 |
| Gastrointestinal disorders | 1 (33%) | 0 | 0 | 0 |
| Nausea | 1 (33%) | 0 | 0 | 0 |
| Musculoskeletal and connective | 1 (33%) | 0 | 0 | 0 |
| tissue disorders | | | | |
| Back pain | 1 (33%) | 0 | 0 | 0 |
| Skin and subcutaneous tissue | 1 (33%) | 1 (25%) | 0 | 0 |
| disorders | | | | |
| Skin abrasion | 1 (33%) | 1 (25%) | 0 | 0 |

Deaths and Serious Adverse Events besides Death

There were no deaths or SAEs during study STUDY A.

### Study Subject Dropouts Due to an Adverse Drug Experience

There were no early discontinuations due to AEs in STUDY A.

Summary of Information Obtained Pertinent to Understanding the Drug's Actions (Dose Response, Bioavailability, Information from Other Controlled Trials, etc.)

Twelve subjects underwent timed blood samplings for PK analysis in STUDY A. All samples collected were below the LLOQ for Formula I (1 ng/mL) and its major metabolite jasmonic acid (50 ng/mL).

In STUDY A, there were no safety related findings that suggested a dose response pattern at the Formula I doses evaluated versus placebo.

### EXAMPLE 4: Phase 2 Study in Subjects with Actinic Keratosis

### Title of Study

A Phase 2 randomized, double-blind, placebo-controlled, parallel-cohort study to evaluate the efficacy, safety, tolerability, and pharmacokinetics of once-daily application of topical Formula I ointment for 28 days in subjects with actinic keratosis.

### Purpose of Study

Safety and efficacy.

### Study Objectives

To compare the reduction on Day 56 in the number of the actinic keratosis (AK) lesions in the Treatment Field of subjects receiving once-daily topical 5% or 10% Formula I ointment for 28 days to the reduction in the number of AK lesions in subjects receiving placebo.

To evaluate the systemic and local (skin) safety and tolerability of once-daily topical application of 5% or 10% Formula I ointment or placebo for 28 days in adult subjects with AK.

To assess the systemic exposure of Formula I and jasmonic acid, its primary metabolite, at selected time points during topical application of 5% or 10% Formula I ointment or placebo for 28 days in adult subjects with AK.

To compare the proportion of subjects having complete clearance of AK lesions on Day 56 between subjects receiving 28 days of 5% or 10% Formula I ointment and subjects receiving placebo.

To compare the reduction on Day 84 in the number of the actinic keratosis (AK) lesions in the Treatment Field of subjects receiving once-daily topical 5% or 10% Formula I ointment for 28 days to the reduction in the number of AK lesions in subjects receiving placebo.

### Subject Population(s) Under Study

Subjects, 18 years and older, who at baseline (Screening and Day 1 pre-dose visits) had a minimum of 4 and a maximum of 8 discrete Grade 1-2 AK lesions within a single 25 cm² area of skin on their scalp or face.

### Treatment Regimen

The Treatment Field is selected at screening and confirmed on Day 1 pre-dose. Any 25 cm² contiguous area on the face or scalp of the subject that contains 4-8 AK lesions that conform to AK lesion eligibility criteria as the study Treatment Field.

Subjects are randomly assigned in a double-blind fashion to 1 of 3 parallel treatment cohorts (5% Formula I ointment, 10% Formula I ointment, or placebo, respectively) in a ratio of 1:1:1. Randomized subjects apply a single, thin application of the study drug to their treatment field for 28 days.

### Subject Enrollment Status

1. Enrollment Time Table

TABLE 8
Enrollment Time Table in the Phase 2 study

| | |
|---|---|
| Date study open to enrollment: | Jul. 28, 2016 |
| Date enrollment closed: | Feb. 2, 2017 |
| Expected end date of study: | Apr. 27, 2017 |

2. Cumulative Enrollment Summary

TABLE 9
Cumulative Enrollment Summary in the Phase 2 study

| | All Subjects^{a} |
|---|---|
| | n (%) |
| Total planned accrual: | 84 (75 evaluable) |

| | | |
|---|---|---|
| Number of subjects enrolled to date ^{b}: | 92 | (100) |
| Number of subjects completed to date ^{b}: | 60 | (65.2) |
| Number of subjects that withdrew/withdrawn | 1 | (1.1) |
| from study to date ^{b} | | |

^{a}Subjects were randomly assigned to 5% or 10% Formula I ointment or placebo.
^{b} Cut-off date of 4 Feb. 2017
One subject withdrew consent on study day 56 due to his extensive winter vacation plans. The subject experienced no AEs during his time enrolled in the study.

3. Total Number of Enrolled Subjects by Age, Gender, and Race

The study has enrolled 82 (89.1%) male patients and 10 (10.9%) female patients. The majority of patients (58.7%) were >65 years of age, and all 92 patients (100%) were Caucasian.

TABLE 10
Number of Enrolled Subjects by Age,
Gender, and Race in the Phase 2 study

| Demographic | | All Subjects^{a} | |
|---|---|---|---|
| parameter | | n (%) | |
| Sex | Female | 10 | (10.9) |
| | Male | 82 | (89.1) |
| Age | 18-29 years | 0 | |
| | 30-49 years | 2 | (2.2) |
| | 50-64 years | 36 | (39.1) |
| | 65-74 years | 38 | (41.3) |
| | 75-84 years | 14 | (15.2) |
| | ≥65 years | 54 | (58.7) |
| | ≥75 years | 16 | (17.4) |
| | ≥85 years | 2 | (2.2) |
| Female by age | 18-29 years | 0 | |
| group | 30-49 years | 0 | |
| | 50-64 years | 5 | (50) |
| | 65-74 years | 3 | (30) |
| | 75-84 years | 2 | (20) |
| | ≥65 years | 5 | (50) |
| | ≥75 years | 2 | (20) |
| | ≥85 years | 0 | |
| Male by age group | 18-29 years | 0 | |
| | 30-49 years | 2 | (2.4) |
| | 50-64 years | 31 | (37.8) |
| | 65-74 years | 35 | (42.7) |
| | 75-84 years | 12 | (14.6) |
| | ≥65 years | 49 | (59.8) |
| | ≥75 years | 14 | (17.1) |
| | ≥85 years | 2 | (2.4) |
| Race | Caucasian | 92 | (100) |
| | Black or African | 0 | |
| | American | | |
| | Asian | 0 | |
| | Native Hawaiian or Other | 0 | |
| | Pacific Islander | | |
| | American Indian or | 0 | |
| | Alaska Native | | |
| | Other | 0 | |
| | Race Not Reported | 0 | |
| Ethnicity | Hispanic or Latino | 0 | |
| | Not Hispanic or Latino | 92 | (100) |

^{a}Subjects were randomly assigned to 5% or 10% Formula I ointment or placebo.

### Phase 2 Study Results

The Phase 2 study is currently ongoing; therefore, the study is still blinded. There have been no deaths or reportable SAEs during the reporting period.

The first 15 (PK and non-PK) subjects enrolled in the trial underwent the safety examinations planned for all other subjects on Day 7 plus physical examination, clinical laboratory and ECG assessments. Once all 15 subjects in this Nested Phase 1b Safety Sub-Cohort completed their Day 7 Visit, safety data (including AEs, Local Skin Reaction (LSR) scores, clinical laboratory results, and ECG data) from Day 1 through Day 7 were reviewed in a blinded fashion by a safety committee consisting of 3 Board-certified internists and sub-specialists. No AEs had been recorded and no safety signal was found. Local skin reactions were unremarkable with no individual LSR above mild.

A single interim efficacy analysis (IEA) was conducted as soon as approximately 50% of the subjects had completed their Day 56 Visit. The objective of the IEA is to evaluate the probability of achieving the primary efficacy endpoint at the conclusion of the study. The unblinded data for the interim analysis sub-set was analyzed by an independent statistician and found that, based upon the data available at the time of the IEA, there was a chance for at least one of the two treatment arms to achieve the primary endpoint.

### Progress Information

The Phase 2 study is being conducted in a target patient population; this study is ongoing, and the treatment code is still blinded.

### Most Frequent and Most Serious Adverse Events

TABLE 11
Serious Adverse Events in the Phase 2 study

| MedDRA System Organ Class | All subjects ^{a} | |
|---|---|---|
| Preferred term | n (%) | |
| Number of subjects enrolled | 92 | (100) |
| Any serious adverse event | 2 | (2.2) |
| Infections and infestations | 1 | (1.1) |
| Pneumonia | 1 | (1.1) |
| Neoplasms benign, malignant and unspecified | 1 | (1.1) |
| (including cysts and polyps) | | |
| Benign sero-mucinous cystadenoma | 1 | (1.1) |

^{a} Subjects were randomly assigned to 5% or 10% Formula I ointment or placebo.

There have been no deaths, suspected unexpected SAEs, study drug related SAEs, or severe AEs reported.

Two SAEs were reported in this trial (Table 11). Neither was deemed related to the study drug by the Investigator or the Sponsor.

The first subject was a 77-year-old female who developed a colo-vaginal fistula in May, 2016 (3 months prior to screening). The subject underwent subsequent laparoscopic sigmoid colectomy on 29 Jun. 2017 at which time a mucinous cystadenoma was incidentally found. The subject scheduled elective out-patient salpingo-oophorectomy for 28 September, but failed to communicate this information to the site personnel at the Screening Visit on 22 August. Subsequently, the subject was enrolled on 30 August and she administered her final study drug application on Day 27 (26 Sep. 2017). At the Day 28 Visit on 27 September, the subject notified the site that she was planning to have elective surgery the next day. Surgery proceeded as planned and was uneventful; however, the subject was hospitalized overnight after the surgeon learned that she would be alone at home overnight. A benign sero-mucinous cystadenoma was reported by the pathologist who examined the specimens removed at surgery. The Investigator and the Sponsor agreed that the SAE was unrelated to the study drug since the surgical condition was diagnosed approximately 2 months prior to randomization.

The second subject was an 81-year-old male with a past medical history of Hemophilia A and asthma who was hospitalized 4 days for treatment of bilateral lower lobe pneumonia during which the study drug was temporarily discontinued. The subject recovered with no sequalae and re-started the study drug upon discharge from the hospital. The Investigator and Sponsor agreed that the SAE was unrelated to the study drug.

### Study Subject Deaths

There have been no deaths reported in the Phase 2 study.

### Study Subject Dropouts Due to an Adverse Drug Experience

One subject discontinued treatment prematurely due to an AE that was judged by the Investigator and Sponsor to be unrelated to the study treatment.

The subject is a 63-year-old white male with a past medical history of coronary heart disease, hypercholesterolemia, diverticulitis, and Fuchs' Corneal Endothelial Dystrophy and whose daily medications were fluorometholone 0.1% eyedrops, aspirin 81 mg orally, and rosuvastatin 10 mg orally. The subject reported mild right eye fatigue on Day 12 and discontinued administration of the study drug on Day 14. The AE ended 2 days later without any intervention or sequelae. Despite discontinuing the study drug, the subject continued his full participation in the trial follow-up and procedures until his scheduled exit. The subject reported 2 additional AEs of constipation and diarrhea on Day 17, three days after discontinuing Formula I ointment, that were mild, self-limited, and were also deemed unrelated to the study drug by the Investigator and Sponsor.

Summary of Information Obtained Pertinent to Understanding the Drug's Actions (Dose Response, Bioavailability, Information from Other Controlled Trials, etc.)

Twelve subjects underwent timed blood samplings for PK analysis in the Phase 2 study. All samples collected were below the LLOQ for Formula I (1 ng/mL) and its major metabolite jasmonic acid (20 ng/mL).

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

1-67. (canceled).

68. A topical pharmaceutical composition comprising a therapeutically effective amount of a compound represented by the structure of Formula I, or a pharmaceutically acceptable salt or solvate thereof, and at least one non-aqueous, pharmaceutically acceptable excipient selected from the group consisting of a glycol diester with a saturated or unsaturated fatty acid, a triglyceride, an aprotic organic solvent, and any combination thereof

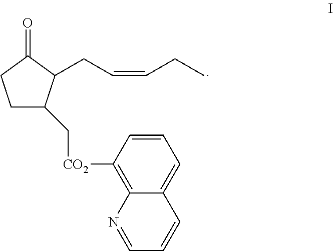

69. The composition according to claim 68, wherein said non-aqueous, pharmaceutically acceptable excipient comprises a compound represented by the structure: 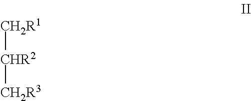
wherein:
R¹, R² and R³ are each selected from the group consisting of H and R⁴—COO—, wherein at least two of R¹, R² and R³ are R⁴—COO; and
R⁴—COO— is the residue of a saturated or unsaturated fatty acid.

70. The composition according to claim 68, wherein said triglyceride comprises C₈-C₁₂ saturated or unsaturated fatty acids, said organic solvent comprises dimethylsulfoxide (DMSO), said non-aqueous, pharmaceutically acceptable excipient comprises caprylic/capric triglyceride, a propylene glycol diester with saturated or unsaturated fatty acids, propylene glycol dicaprylate, or a combination thereof.

71. The composition according to claim 70, wherein each of said fatty acids is a C₈-C12 saturated or unsaturated fatty acid.

72. The composition according to claim 68, further comprising an oil, a wax, or a combination thereof.

73. The composition according to claim 68, comprising a compound of Formula I in an amount of from about 5% w/w to about 30% w/w.

74. The composition according to claim 68, comprising a compound of Formula I, propylene glycol dicaprylate, caprylic/capric triglyceride, dimethylsulfoxide (DMSO), petrolatum and paraffin wax.

75. The composition according to claim 74, comprising:
a compound of Formula I in an amount of about 5% to 30% w/w;
propylene glycol dicaprylate in an amount of about 1% to about 10% w/w;
caprylic/capric triglyceride in an amount of about 30% to about 50% w/w;
dimethyl sulfoxide in an amount of about 0% to about 10% w/w;
petrolatum in an amount of about 20% to about 50% w/w; and
paraffin wax in an amount of about 0 to about 10% w/w.

76. The composition according to claim 75, comprising:
a compound of Formula I in an amount of about 5% w/w;
propylene glycol dicaprylate in an amount of about 10% w/w;
caprylic/capric triglyceride in an amount of about 50% w/w;
dimethyl sulfoxide in an amount of about 1% w/w;
petrolatum in an amount of about 30% w/w; and
paraffin wax in an amount of about 4% w/w
or
a compound of Formula I in an amount of about 10% w/w;
propylene glycol dicaprylate in an amount of about 7.5% w/w;
caprylic/capric triglyceride in an amount of about 47.5% w/w;
dimethyl sulfoxide in an amount of about 1% w/w;
petrolatum in an amount of about 30% w/w; and
paraffin wax in an amount of about 4% w/w.
or
a compound of Formula I in an amount of about 15% w/w;
propylene glycol dicaprylate in an amount of about 5% w/w;
caprylic/capric triglyceride in an amount of about 45% w/w;
dimethyl sulfoxide in an amount of about 1% w/w;
petrolatum in an amount of about 30% w/w; and
paraffin wax in an amount of about 4% w/w.
or
a compound of Formula I in an amount of about 20% w/w;
propylene glycol dicaprylate in an amount of about 2.5% w/w;
caprylic/capric triglyceride in an amount of about 42.5% w/w;
dimethyl sulfoxide in an amount of about 1% w/w;
petrolatum in an amount of about 30% w/w; and
paraffin wax in an amount of about 4% w/w.

77. The composition according to claim 68, in the form of an ointment, cream or gel.

78. The composition according to claim 68, wherein said composition is essentially free of water, alcoholic solvents and carboxylic acids.

79. A method of treating or preventing cancer or a pre-cancerous condition, or a benign hyperproliferative disorder, the method comprising the step of administering to a subject in need thereof a pharmaceutical composition comprising a therapeutically effective amount of a compound represented by the structure of Formula I, 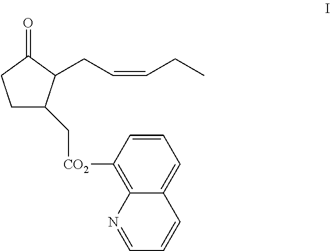
or a pharmaceutically acceptable salt or solvate thereof, and at least one non-aqueous, pharmaceutically acceptable excipient selected from the group consisting of a glycol diester with a saturated or unsaturated fatty acid, a triglyceride, an aprotic organic solvent, and any combination thereof.

80. The method according to claim 79, wherein the composition comprises:
a compound of Formula I in an amount of about 5% to 30% w/w;
propylene glycol dicaprylate in an amount of about 1% to about 10% w/w;
caprylic/capric triglyceride in an amount of about 30% to about 50% w/w;
dimethyl sulfoxide in an amount of about 0% to about 10% w/w;
petrolatum in an amount of about 20% to about 50% w/w; and
paraffin wax in an amount of about 0 to about 10% w/w.

81. The method according to claim 80, wherein the composition comprises:
a compound of Formula I in an amount of about 5% w/w;
propylene glycol dicaprylate in an amount of about 10% w/w;
caprylic/capric triglyceride in an amount of about 50% w/w;
dimethyl sulfoxide in an amount of about 1% w/w;
petrolatum in an amount of about 30% w/w; and
paraffin wax in an amount of about 4% w/w.
or
a compound of Formula I in an amount of about 10% w/w;
propylene glycol dicaprylate in an amount of about 7.5% w/w;
caprylic/capric triglyceride in an amount of about 47.5% w/w;
dimethyl sulfoxide in an amount of about 1% w/w;
petrolatum in an amount of about 30% w/w; and
paraffin wax in an amount of about 4% w/w.
or
a compound of Formula I in an amount of about 15% w/w;
propylene glycol dicaprylate in an amount of about 5% w/w;
caprylic/capric triglyceride in an amount of about 45% w/w;
dimethyl sulfoxide in an amount of about 1% w/w;
petrolatum in an amount of about 30% w/w; and
paraffin wax in an amount of about 4% w/w.
or
a compound of Formula I in an amount of about 20% w/w;
propylene glycol dicaprylate in an amount of about 2.5% w/w;
caprylic/capric triglyceride in an amount of about 42.5% w/w;
dimethyl sulfoxide in an amount of about 1% w/w;
petrolatum in an amount of about 30% w/w; and
paraffin wax in an amount of about 4% w/w.

82. The method according to claim 79, wherein said condition or disorder is psoriasis or actinic keratosis.

83. The method according to claim 79, wherein said cancer is skin cancer.

84. The method according to claim 83, wherein said skin cancer comprises melanoma or a non-melanoma skin cancer.

85. The method according to claim 84, wherein said non-melanoma skin cancer comprises basal cell carcinoma (BCC) or squamous cell carcinoma (SCC) or cutaneous T-cell lymphoma (CTCL).

86. The method according to claim 79, wherein said composition is administered in combination with at least one other active agent.

87. The method according to claim 79, wherein said composition is administered one, two, three, or four times per day, or once every 2 days, once every 3 days, once every 4 days, once every 5 days, once every 6 days, once every 7 days, once every 8 days, once every 9 days, or once every 10 days.
